(19) United States
(12) Patent Application Publication
(10) Pub. No.: US 2012/0008091 A1
(43) Pub. Date: Jan. 12, 2012

(54) EVALUATING PUPILLARY RESPONSES TO LIGHT STIMULI

(76) Inventor: Charles W. Stewart, La Quinta, CA (US)

(21) Appl. No.: 13/177,164

(22) Filed: Jul. 6, 2011

Related U.S. Application Data
(60) Provisional application No. 61/361,535, filed on Jul. 6, 2010.

(51) Int. Cl. A61B 3/14; A61B 3/00

(52) U.S. Cl. 351/206; 351/246

(57) ABSTRACT

A solution for evaluating the pupillary responses of a patient is disclosed. The solution includes exposing one eye of a patient to flashes having varying patterns and concurrently recording the pupillary responses of both eyes of the patient to the flash. At least some of the patterns can be configured to stimulate/not stimulate the macula region of the patient's eye. The solution also can include reducing one or more non-visual stimuli experienced by the patient while the pupillary responses are being induced and recorded.

### REFERENCE TO RELATED APPLICATION

The current application claims the benefit of co-pending U.S. Provisional Application No. 61/361,535, titled “Evaluating Pupillary Responses to Light Stimuli,” which was filed on Jul. 6, 2010, and which is hereby incorporated by reference.

### TECHNICAL FIELD

The invention generally relates to a solution for acquiring data for evaluating pupillary responses to light stimuli. More particularly, an embodiment of the invention provides a solution for exposing one or both eyes to a series of light flashes having varying patterns and recording data corresponding to the direct and/or consensual pupillary reflexes that can be used to detect the presence of various dysfunctions and/or conditions.

### BACKGROUND ART

During eye examinations, the pupillary reflexes of a patient are often monitored to determine the presence of various ocular dysfunctions. The presence of one or more ocular dysfunctions can signal that the patient suffers from an ocular disorder such as optic neuropathy, other pathology of the ocular pathways between the photoreceptors of the retina and brain, opacification of the ocular media, or conditions that impact the transmission of light through the ocular media. A common objective visual functional test for the detection of such visual dysfunctions is the “Swinging Flashlight Test” (SFT). For the SFT, a handheld, very bright light source is shined first into one eye of the patient and then into the other eye, in a pendular fashion with a period of one to two seconds. While this is being done, the examiner will observe the reflexes of the patient's pupils. A detection of a positive sign is made based on the observed reflexes.

For example, if the light is shined into an eye that has an optic nerve conduction defect, while the other eye does not, the pupil of the eye with the defect will contract to a lesser degree than will the pupil of the eye without the defect when that eye is stimulated with the same light. Similarly, if both eyes have a defect, one having a greater defect than the other, the light being shined into the eye with the greater defective optic nerve will evoke a lesser pupillary contraction of both pupils than would the same light shined into the eye with the lesser optic nerve defect, thus yielding a sign of a Relative Afferent Pupillary Defect (RAPD). Moreover, in the presence of a RAPD, when the light is alternated every few seconds between the two eyes, these differences in pupillary reflexes to the same bright light shined into the two eyes can give rise to an “illusion” that shining the same bright light into the eye with the greater defect caused its pupil to dilate (or expand), a so called Marcus-Gunn pupil.

Clinical methods currently exist to quantify RAPD by a nullifying exercise in which the light entering the more sensitive eye is reduced by placing Neutral Density (ND) filters, sbisa bars, or crossed polarizing filters between the eye and the light in increasing grades until the positive sign of pupillary escape is no longer apparent. The intensity of the filter used to reach this nullification can be viewed as a quantitative measurement of the difference in light sensitivity between the two eyes, and is usually expressed in Log units of ND. Generally, RAPDs of a magnitude less than 0.3 Log units are not measurable with this procedure, possibly owing to the limitations of the examiner being able to observe small changes in pupil size of both eyes simultaneously. Often, 0.3 Log units is the minimum magnitude ND filter available to clinicians.

The SFT is an example of an objective functional test of the visual system that depends upon differences in pupillary reflexes to infer the presence of an ocular dysfunction. The presence of an ocular dysfunction can indicate an ocular disorder (i.e., disease or pathology). However, the SFT has numerous drawbacks. In particular, it lacks specificity for any one ocular disorder whether of neurological or transmissive origin. For example, it can be positive in unilateral dense cataracts, in certain unilateral retinal disorders, in anisocoria, as well as in significant asymmetric glaucoma. A clinician performing the SFT cannot tell which ocular disorder is present based solely on the pupillary reflexes. Moreover, the SFT lacks sensitivity due to the manner in which the differences between the direct and consensual reflexes are observed. For example, the clinician cannot observe the pupils of both eyes simultaneously, but must visualize the reflex of one pupil first and then visualize the reflex of the other pupil moments later. As a result, small differences in reflexes may go unnoticed. The unaided observation makes this comparative judgment subject to significant error and makes the detection of small differences in reflexes between the two eyes especially problematic. Because the SFT relies on the examiner's naked eye to detect and diagnose ocular dysfunctions, it lacks practical utility. Moreover, by depending on a single bright light, the SFT stimulates the visual system in an indiscriminate manner. As a result, this manner of evoking the pupils' reflexes is lacking in both sensitivity and specificity.

Further, several observations have been made concerning the ocular disorder glaucoma, thought to be a form of optic neuropathy. First, patients with glaucoma and patients present with symptoms of glaucoma display a significant degree of dyschromatopsia, i.e., deficiencies in color discrimination. Second, patients with asymmetric glaucoma, as measured by visual field loss and cup-disc ratios, manifest gross afferent pupillary defects to a greater extent than do patients without glaucoma. Third, a consensual pupillary reflex can be induced by the interchange of equally luminous, heterochromatic members of a pair of lights shined into the patient's contralateral eye. This finding indicates that chromatic differences in stimuli activate pupillary reflexes via stimulation of different cell populations, and that such activation is independent of the luminosity change thought to be the primary basis of pupillary reflex activation in the SFT.

Attempts have been made to solve these problems by implementing systems and devices for measuring pupillary reflexes to light stimuli. Such devices generally implement a system for exposing a patient's eyes to stimuli and then measuring the pupillary reaction thereof. One solution uses a series of flashes that vary chromatically, by luminosity, and/or by location in the visual field. Regardless, the goal is to intentionally induce a pupillary reflex and then measure the reflex using various means. Because dimensional changes in the pupil's movements often can be minuscule, the comparison to a range of “normal” reactions obtained from different patients can lack accuracy. Without an appropriate validation procedure, this could lead to a false diagnosis of a disorder that is not present, a failure to diagnose a disorder that is present, or a failure to distinguish between two ocular diseases. Furthermore, if the examiner is seeking specific information, for example, about the afferent optic nerve pathology of a patient, efferent deficiencies may significantly confound the interpretation of such information.

### SUMMARY OF THE INVENTION

Aspects of the invention provide a solution for evaluating the pupillary responses (e.g., reflexes) of a patient. The solution includes exposing one eye of a patient to flashes having varying patterns and concurrently recording the pupillary responses of both eyes of the patient to the flash. At least some of the patterns can be configured to stimulate/not stimulate the macula region of the patient's eye. The solution also can include reducing one or more non-visual stimuli experienced by the patient while the pupillary responses are being induced and recorded.

A first aspect of the invention provides a method comprising: exposing a first eye of a patient to a series of flashes, wherein the series of flashes includes at least one flash corresponding to each of a plurality of patterns, wherein each of the plurality of patterns is configured to stimulate at least one of: a targeted visual function or a targeted cell population of a retina of the first eye; and concurrently acquiring image data corresponding to the pupillary reflexes of the first eye and a second eye of the patient during the exposing.

A second aspect of the invention provides a system comprising: a stimulus panel configured to provide a flash of light having any one of a plurality of patterns; a set of imaging devices configured to concurrently acquire image data of a first eye and a second eye of a patient; and a computer system for performing a method comprising: exposing the first eye of the patient to a series of flashes using the stimulus panel, wherein the series of flashes includes at least one flash corresponding to each of the plurality of patterns, wherein each of the plurality of patterns is configured to stimulate at least one of: a targeted visual function or a targeted cell population of a retina of the first eye; and concurrently acquiring image data corresponding to the pupillary reflexes of the first eye and the second eye of the patient during the exposing using the set of imaging devices.

A third aspect of the invention provides a system for evaluating a patient, the system comprising: a stimulus panel configured to generate a flash of light having any one of a plurality of patterns; a set of imaging devices configured to concurrently acquire image data of a first eye and a second eye of a patient; and a computer system for performing a method comprising: exposing the first eye of the patient to a series of flashes using the stimulus panel, wherein the series of flashes includes at least one flash corresponding to each of the plurality of patterns, wherein each of the plurality of patterns is configured to stimulate at least one of: a targeted visual function or a targeted cell population of a retina of the first eye; concurrently acquiring image data corresponding to the pupillary reflexes of the first eye and the second eye of the patient during the exposing using the set of imaging devices; repeating the exposing and concurrently acquiring for the second eye of the patient; and determining a pupillary response of the first eye and the second eye to each of the series of flashes corresponding to the first eye and the second eye.

Other aspects of the invention provide methods, systems, program products, and methods of using and generating each, which include and/or implement some or all of the actions described herein. The illustrative aspects of the invention are designed to solve one or more of the problems herein described and/or one or more other problems not discussed.

### BRIEF DESCRIPTION OF THE DRAWINGS

These and other features of this invention will be more readily understood from the following detailed description of the various aspects of the invention taken in conjunction with the accompanying drawings that depict various aspects of the invention.

FIG. 1 shows an illustrative system for evaluating pupillary responses of a patient according to an embodiment.

FIGS. 2A-2B show side and front views, respectively, of an illustrative pupillary imaging device according to an embodiment.

FIG. 3 shows an illustrative process for imaging the pupils of a patient according to an embodiment.

FIG. 4 shows an illustrative graph corresponding to the diameter of each pupil of a patient during an illustrative series of stimuli according to an embodiment.

FIGS. 5A-5E show illustrative stimulus patterns according to an embodiment.

It is noted that the drawings of the invention are not necessarily to scale. The drawings are intended to depict only typical aspects of the invention, and therefore should not be considered as limiting the scope of the invention. In the drawings, like numbering represents like elements between the drawings.

### DETAILED DESCRIPTION OF THE INVENTION

As indicated above, aspects of the invention provide a solution for evaluating the pupillary responses (e.g., reflexes) of a patient. The solution includes exposing one eye of a patient to flashes having varying patterns and concurrently recording the pupillary responses of both eyes of the patient to the flash. At least some of the patterns can be configured to stimulate/not stimulate the macula region of the patient's eye. The solution also can include reducing one or more non-visual stimuli experienced by the patient while the pupillary responses are being induced and recorded. As used herein, unless otherwise noted, the term “set” means one or more (i.e., at least one) and the phrase “any solution” means any now known or later developed solution.

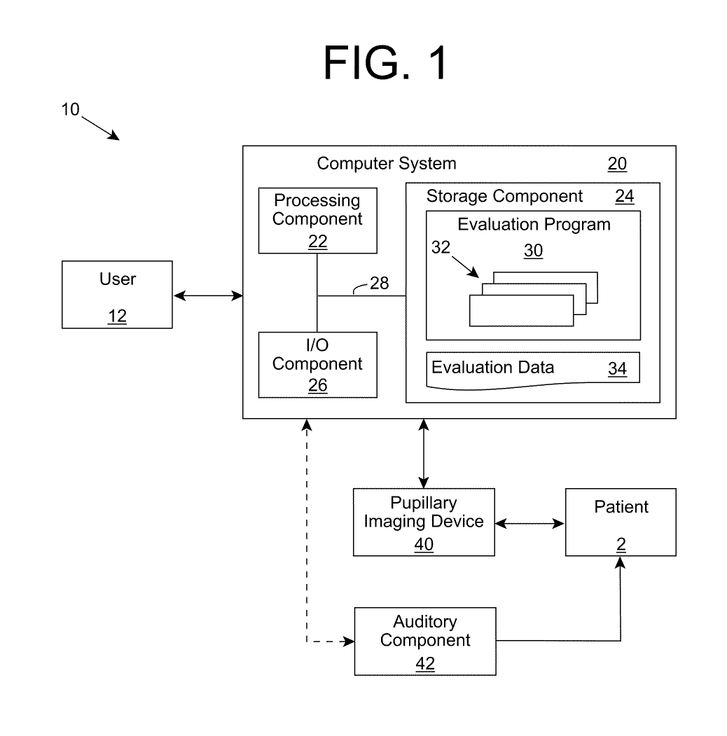

Turning to the drawings, FIG. 1 shows an illustrative pupillary evaluation system 10 for evaluating pupillary responses (e.g., reflexes) of a patient 2 according to an embodiment. To this extent, pupillary evaluation system 10 includes a computer system 20 that can perform a process described herein in order to record and/or evaluate pupillary responses of the patient 2 to a series of stimuli. In particular, computer system 20 is shown including an evaluation program 30, which makes computer system 20 operable to record and/or evaluate pupillary responses of the patient 2 to a series of stimuli by performing a process described herein.

Computer system 20 is shown including a processing component 22 (e.g., one or more processors), a storage component 24 (e.g., a storage hierarchy), an input/output (I/O) component 26 (e.g., one or more I/O interfaces and/or devices), and a communications pathway 28. In general, processing component 22 executes program code, such as evaluation program 30, which is at least partially fixed in storage component 24. While executing program code, processing component 22 can process data, which can result in reading and/or writing transformed data from/to storage component 24 and/or I/O component 26 for further processing. Pathway 28 provides a communications link between each of the components in computer system 20. I/O component 26 can comprise one or more human I/O devices, which enable a human user 12 to interact with computer system 20 and/or one or more communications devices to enable a system user 12 to communicate with computer system 20 using any type of communications link. To this extent, computer system 20 can manage a set of interfaces (e.g., graphical user interface(s), application program interface, and/or the like) that enable human and/or system users 12 to interact with evaluation program 30. Further, computer system 20 can manage (e.g., store, retrieve, create, manipulate, organize, present, etc.) the data, such as evaluation data 34, using any solution.

In any event, computer system 20 can comprise one or more general purpose computing articles of manufacture (e.g., computing devices) capable of executing program code, such as evaluation program 30, installed thereon. As used herein, it is understood that “program code” means any collection of instructions, in any language, code or notation, that cause a computing device having an information processing capability to perform a particular action either directly or after any combination of the following: (a) conversion to another language, code or notation; (b) reproduction in a different material form; and/or (c) decompression. To this extent, evaluation program 30 can be embodied as any combination of system software and/or application software.

Furthermore, evaluation program 30 can be implemented using a set of modules 32. In this case, a module 32 can enable computer system 20 to perform a set of tasks used by evaluation program 30, and can be separately developed and/or implemented apart from other portions of evaluation program 30. As used herein, the term “component” means any configuration of hardware, with or without software, which implements the functionality described in conjunction therewith using any solution, while the term “module” means program code that enables a computer system 20 to implement the actions described in conjunction therewith using any solution. When fixed in a storage component 24 of a computer system 20 that includes a processing component 22, a module is a substantial portion of a component that implements the actions. Regardless, it is understood that two or more components, modules, and/or systems may share some/all of their respective hardware and/or software. Additionally, it is understood that some of the functionality discussed herein may not be implemented or additional functionality may be included as part of computer system 20.

When computer system 20 comprises multiple computing devices, each computing device can have only a portion of evaluation program 30 fixed thereon (e.g., one or more modules 32). However, it is understood that computer system 20 and evaluation program 30 are only representative of various possible equivalent computer systems that may perform a process described herein. To this extent, in other embodiments, the functionality provided by computer system 20 and evaluation program 30 can be at least partially implemented by one or more computing devices that include any combination of general and/or specific purpose hardware with or without program code. In each embodiment, the hardware and program code, if included, can be created using standard engineering and programming techniques, respectively.

Regardless, when computer system 20 includes multiple computing devices, the computing devices can communicate over any type of communications link. Furthermore, while performing a process described herein, computer system 20 can communicate with one or more other computer systems using any type of communications link. In either case, the communications link can comprise any combination of various types of optical fiber, wired, and/or wireless links; comprise any combination of one or more types of networks; and/or utilize any combination of various types of transmission techniques and protocols.

As discussed herein, evaluation program 30 enables computer system 20 to record and/or evaluate pupillary responses of a patient 2 to a series of stimuli using a process described herein. In general, patient 2 can comprise any type of animal having one or more pupils that react to various light stimuli. In an embodiment, the patient 2 is a human. While the term “patient” is used herein to refer to the human/animal whose pupils are being imaged, it is understood that the stimulation and imaging of the pupillary responses of a human/animal can be performed for any purpose, medical or non-medical.

Computer system 20 can operate a pupillary imaging device 40, which can generate various light stimuli and record the corresponding pupillary responses to the light stimuli using any solution. Each light stimulus can be provided to one pupil of the patient 2, and pupillary imaging device 40 can concurrently record the responses of both pupils of the patient 2 to the stimulus. Subsequently, computer system 20 can process the recorded pupillary responses and store data corresponding to the pupillary responses as evaluation data 34 for the patient 2. The evaluation data 34 can include various data corresponding to the light stimuli, recorded responses, and/or the like. Furthermore, the evaluation data 34 can include the raw data recorded by pupillary imaging device 40. In an embodiment, computer system 20 and pupillary imaging device 40 comprise a machine vision system capable of recording image data of the pupils of a patient 2 and tracking the movement of the pupils.

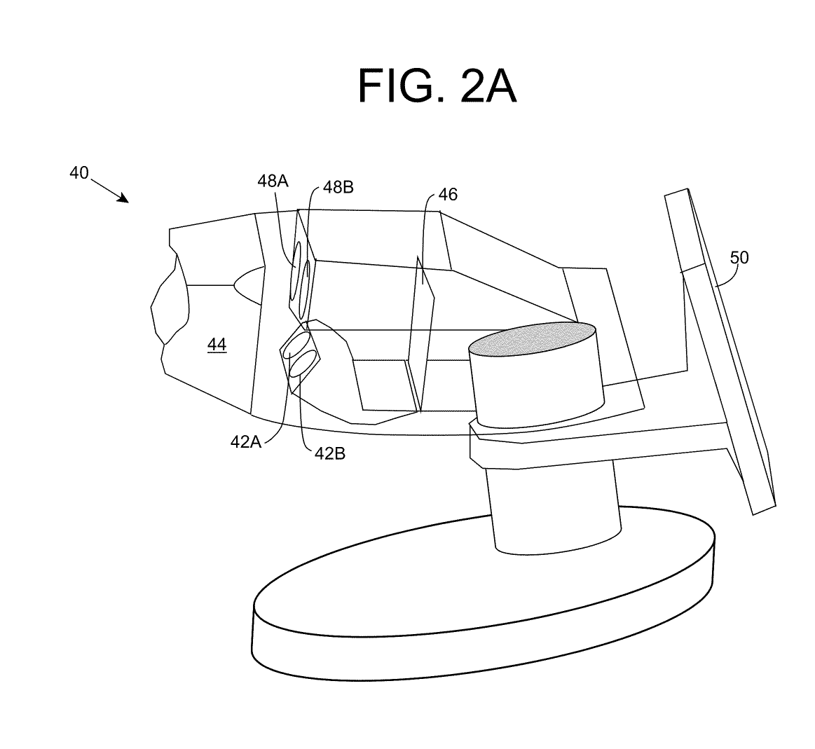

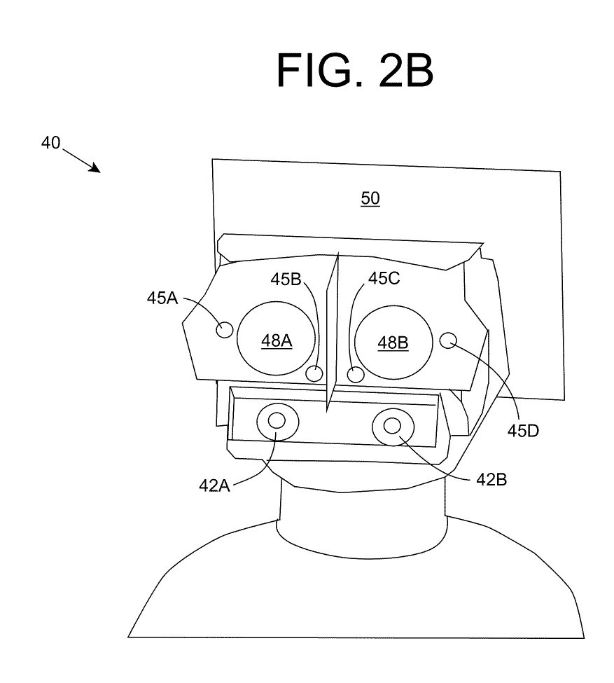

FIGS. 2A-2B show side and front views, respectively, of various components of an illustrative pupillary imaging device 40 according to an embodiment. Pupillary imaging device 40 includes a pair of imaging devices 42A, 42B, each of which is configured to record image data corresponding to a unique pupil of a patient 2 (FIG. 1), whose eyes are located adjacent to a facemask 44. Facemask 44 can be configured to limit interference from any extraneous light with the imaging of the patient's 2 pupils by the imaging devices 42A, 42B. To this extent, pupillary imaging device 40 can include a set of pupil illuminating devices 45A-45D. As illustrated, the pupil illuminating devices 45A-45D can be mounted at approximately a thirty-five degree angle from the imaging devices 42A, 42B and the pupil of the patient 2 to enable improved discrimination of the pupil by computer system 20. Each pupil illuminating device 45A-45D can be configured to illuminate a pupil of the patient 2 with electromagnetic radiation conducive for acquiring bilateral image data corresponding to the pupils, which can be accurately evaluated by the computer system 20. In an embodiment, each pupil illuminating device 45A-45D emits infrared light, e.g., having a peak emission wavelength of approximately 880 nanometers, which is imaged by imaging devices 42A, 42B. In a more specific embodiment, each pupil illuminating device 45A-45D comprises an infrared light emitting diode. While pupillary imaging device 40 is shown including two pairs of pupil illuminating devices 45A-45D for illuminating the pupils of a patient 2, it is understood that any number of pupil illuminating devices 45A-45D located in any suitable location(s) can be utilized.

Imaging devices 42A, 42B can acquire image data using any solution. To this extent, imaging devices 42A, 42B can record image data based on infrared light, visible light, and/or ultraviolet light. In an embodiment, imaging devices 42A, 42B are configured to capture image data corresponding to infrared light emitted by pupil illuminating devices 45A-45D. Furthermore, imaging devices 42A, 42B can record a series of images as video data with a corresponding frame rate of at least tens of frames per second. In an embodiment, each imaging device 42A, 42B can acquire image data based on infrared light at a rate of approximately forty hertz, a field of view resolution of approximately 240×240 pixels per frame, and approximately 23 pixels per millimeter resolution of the pupil of the patient 2. While a pair of imaging devices 42A, 42B are shown, one for each pupil of the patient 2, it is understood that any number of one or more imaging devices 42A, 42B can be utilized for acquiring image data corresponding to the pupils of a patient 2.

Pupillary imaging device 40 can be configured to generate light stimuli, which are configured to invoke a response (e.g., reflex) by the pupils of the patient 2. To this extent, pupillary imaging device 40 is further shown including a stimulus panel 46, and a pair of lenses 48A, 48B. Stimulus panel 46 can comprise any type of panel for providing a light stimulus to one or both pupils of the patient 2. In an embodiment, stimulus panel 46 comprises a liquid crystal display (LCD) panel. In a more particular embodiment, the stimulus panel 46 comprises an LCD panel configured to produce light having white, red (e.g., having a peak wavelength of approximately 605 nanometers), green (e.g., having a peak wavelength of approximately 555 nanometers), yellow (e.g., having a peak wavelength of approximately 576 nanometers), and blue (e.g., having a peak wavelength of approximately 440 nanometers) colors both as a full field stimulus and as a specific pattern. As used herein, the term “white” light can refer to light comprising a combination of two or more wavelengths, which when combined, appear to be white, e.g., light having a CIE color temperature of approximately 5500K. The lenses 48A, 48B can be configured to adjust (e.g., expand) the light stimulus on the stimulus panel 46 to a desired size of the visual field of the patient 2. In this manner, the stimulus panel 46 can be smaller and/or located further away from the patient 2 than would otherwise be required. It is understood that pupillary imaging device 40 is only illustrative. To this extent, alternative configurations for imaging pupils, generating stimuli, illuminating the pupils, and/or the like, can be utilized. Furthermore, it is understood that pupillary imaging device 40 can include other components, which are not shown for clarity, such as mirrors, filters, support structures, adjustment mechanisms, and/or the like, to implement the desired illumination, stimulation, and/or imaging of the pupils of various patients 2.

In operation, computer system 20 (FIG. 1) can operate pupillary imaging device 40 to present a series of light stimuli to the eyes (e.g., pupils) of a patient 2 and record image data corresponding to the pupils of the patient 2 in response to the light stimuli. Pupillary imaging device 40 can include synchronization circuitry that synchronizes the time during which a stimulus is presented to an eye with the image data captured by the imaging devices 42A, 42B. The inclusion of synchronization circuitry can improve/optimize the measurement and/or evaluation of spatial (e.g., pupil dimension data), temporal (e.g., latency data), and/or other pupil response metrics as a result of each stimulus. A user 12 (FIG. 1), such as a clinician, can ensure that the patient 2 is appropriately positioned, and select one of a plurality of tests to be performed on the patient 2 using the pupillary imaging device 40. To this extent, pupillary imaging device 40 can include an operator interface 50, such as an LCD control panel or the like, which enables the user 12 to direct operation of the pupillary imaging device 40 by the computer system 20, review image data and/or results of a test, preview image data that will be acquired by imaging devices 42A, 42B, and/or the like.

Furthermore, computer system 20 can automatically determine when a patient 2 is appropriately positioned. To this extent, facemask 44 can include one or more sensors, which can be configured to indicate when the patient 2 is properly positioned using any solution. For example, the sensors can include pressure sensors that activate when the patient's 2 head is pressing against the sensor location. Similarly, computer system 20 can analyze preliminary image data acquired by imaging devices 42A, 42B to determine when the patient 2 is appropriately positioned for imaging during a test, e.g., by identifying the location of the pupils within the image data.

In an embodiment, computer system 20 includes a first computing device collocated with the pupillary imaging device 40, which is operatively connected to the various components of pupillary imaging device 40. For example, the first computing device can be operatively connected to the pupillary imaging device 40 using a local, physical connection, e.g., using a universal serial bus (USB) connection, embedded within the pupillary imaging device 40, connected using a local network, and/or the like. The first computing device can enable a user 12 to perform various operations using pupillary imaging device 40 and computer system 20, such as providing information on patient 2, making one or more adjustments to the pupillary imaging device 40 (e.g., adjusting a region of interest for the image capture), selecting a series of stimuli to be utilized, acquiring image data and generating evaluation data 34 based thereon, and/or the like. Additionally, computer system 20 can include a second computing device, which is located remote from the pupillary imaging device 40 and is in communication with the first computing device over a public and/or private network, such as the Internet. The second computing device can perform analysis and/or evaluation of the evaluation data 34 and/or the patient 2, and provide the results of the analysis for use by the user 12 and/or patient 2.

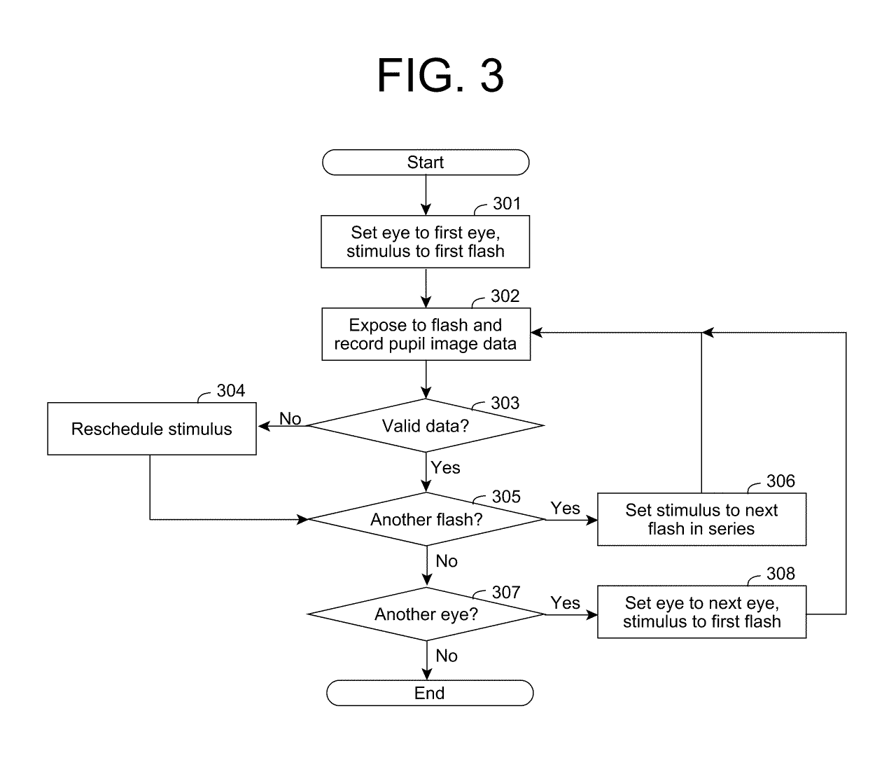

As discussed herein, computer system 20 can operate pupillary imaging device 40 to present a series of stimuli to the pupils of a patient 2 and concurrently record image data corresponding to the response of the pupils to each stimuli, e.g., as part of an evaluation of the patient 2. To this extent, FIG. 3 shows an illustrative process for imaging the pupils of a patient 2, which can be implemented by computer system 20 (FIG. 1), according to an embodiment. In process 301, computer system 20 can configure pupillary imaging device 40 to stimulate a first eye of the patient 2 with a first stimulus in a series of stimuli, e.g., a first flash in a series of flashes. As described herein, a series of flashes can include a plurality of flashes, in which each flash differs from every other flash by one or more of: pattern, luminosity, size, location in the visual field, color, and/or the like.

Regardless, in process 302, computer system 20 can operate pupillary imaging device 40 to expose the first eye of the patient 2 to the current stimulus, e.g., the current flash in the series of flashes, and concurrently record pupil image data for both of the pupils of the patient 2. While the first eye is exposed to the stimulus, the second eye does not receive any stimulus. In an embodiment, computer system 20 operates the pupillary imaging device 40 to record the pupils of the patient 2 for a time frame that includes at least a time just prior to the stimulus and lasts for a duration of time after the stimulus. The duration can be selected to at least include the expected time required for the pupils to recover to the same state as prior to exposure to the stimulus (e.g., the flash).

In process 303, computer system 20 can analyze the image data and determine whether it is valid. For example, computer system 20 can determine whether the pupils remain visible and are measurable for a sufficient amount of time, whether a blink occurred during the recording, and/or the like. When computer system 20 determines that the image data is valid, computer system 20 can store the image data and/or data corresponding to the image data as evaluation data 34 (FIG. 1). Otherwise, in process 304, computer system 20 can reschedule the stimulus for re-exposing the pupil. The stimulus can be rescheduled to any time, e.g., to immediately occur as the next flash, occur at the end of the series of scheduled flashes for the particular eye, occur after all other regularly scheduled flashes for either eye have occurred, and/or the like.

In process 305, computer system 20 can determine whether there is another stimulus, e.g., flash, in the current series of stimuli. If so, in process 306, computer system 20 can configure the pupillary imaging device 40 to generate the next stimulus (e.g., flash) in the series, and the process can return to process 302. Otherwise, in process 307, computer system 20 can determine whether another eye of the patient 2 is to be stimulated. This can include presenting a series of stimuli to a second eye of the patient 2, re-stimulating the previous eye with rescheduled stimuli, and/or the like. If so, in process 308, computer system 20 can configure the pupillary imaging device 40 to generate the first stimulus (e.g., flash) in the series of stimuli for the next eye of the patient 2, and the process can return to process 302. Otherwise, the process can terminate, after which the computer system 20 will comprise evaluation data 34 corresponding to the series of stimuli for the desired eye(s) of the patient 2.

As described herein, a series of stimuli can include a series of flashes. In an embodiment, each flash comprises a beam of light having a short time duration. In a more particular embodiment, each flash is terminated before the release (escape) phase of the pupillary reflex has begun. This release phase can take, for example, approximately 0.6 seconds. In an illustrative series of flashes, each flash comprises a duration of approximately 0.6 seconds and is followed by a recovery period of approximately 2.4 seconds during which no stimulus is provided to the pupils of the patient 2. However, it is understood that this series of flashes is only illustrative, and flashes and/or recovery periods of shorter/longer can be utilized.

During the series of stimuli, computer system 20 can operate pupillary imaging device 40 to assist the patient 2 in directing his/her view towards a desired location/direction. For example, pupillary imaging device 40 can generate a fixation target, such as a green cross, on the stimulus panel 46 against a background illuminance of approximately one lux. Furthermore, as described herein, computer system 20 can evaluate the image data acquired during the series of stimuli to determine whether any stimulus requires rescheduling. In particular, computer system 20 can dynamically determine whether the pupil in each image can be evaluated. For example, computer system 20 can: determine whether the pupil is out of round by more than an acceptable margin, thereby indicating that the patient's view is misdirected; determine whether the pupil is obscured, e.g., due to a blink, ambient light, or the like; and/or the like.

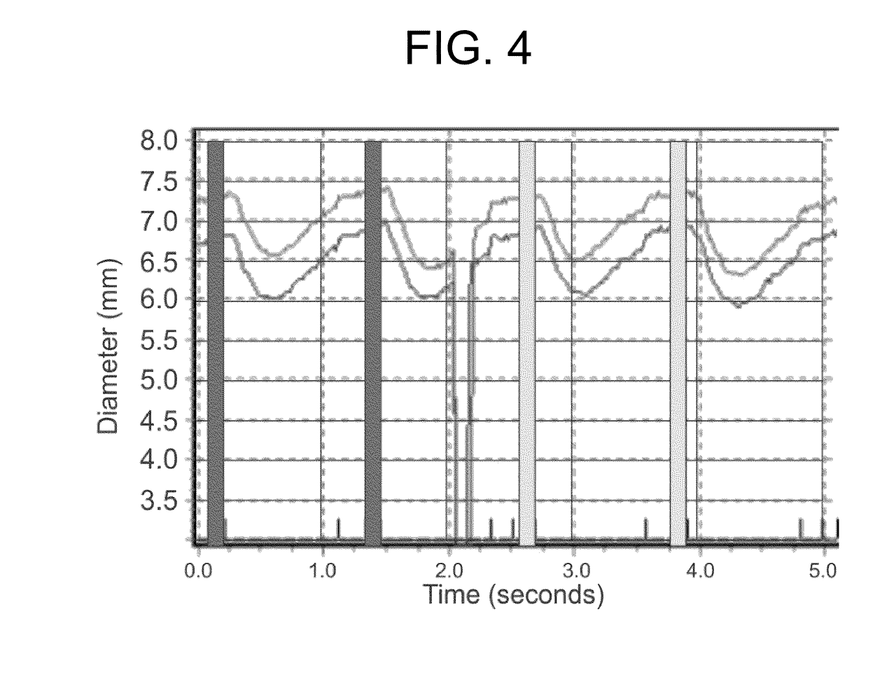

Computer system 20 can process the image data to generate evaluation data 34 representative of the bilateral responses of the pupils to each of the stimuli. For example, FIG. 4 shows an illustrative graph corresponding to the diameter of each pupil of a patient 2 during an illustrative series of stimuli, which can be generated by computer system 20, according to an embodiment. In particular, computer system 20 can process each image of each eye and calculate a diameter of the corresponding pupils using any solution. Subsequently, the calculated diameters can be correlated with a time for the corresponding frame and plotted. As illustrated in the graph, the calculated diameters of both pupils can be plotted together, e.g., using a different color, which allow for ready comparison of the responses of the pupils to each stimulus. In the plot shown in FIG. 4, the vertical bars indicate times during which pupillary imaging device 40 is exposing one of the pupils to the stimulus. Furthermore, as illustrated in the plot, an eye blink occurred around two seconds into the plot, which computer system 20 can identify (e.g., since the calculated diameters went to zero). In response, to identifying the eye blink, computer system 20 can determine whether the eye blink occurred during a critical segment of the pupil responses elicited by the stimulus. If so, computer system 20 can automatically reschedule the stimulus that was previously presented. Otherwise, computer system 20 can use the remaining evaluation data 34 to evaluate the pupillary responses to the stimulus.

In an embodiment, a series of flashes includes flashes having a plurality of shapes/patterns. Each pattern can be configured to stimulate one or more different visual functions and/or cell populations of the retina. Computer system 20 and pupillary imaging device 40 can implement an eye tracking process to monitor and guide (e.g., aim) the light of each pattern onto the retina of the patient 2.

In a further embodiment, the patterned flashes can comprise any of various elliptical (e.g., circular) patterns, annular/semi-annular patterns, and/or the like. In a more particular embodiment, the elliptical pattern is circular, while the annular pattern can be either a full annulus or a portion of an annulus. The patterns can be configured based on a size and/or location of a macula of the corresponding eye being exposed. In particular, a first pattern can be configured to approximate an area of the macula, while a second pattern can be configured to specifically exclude the macular area.

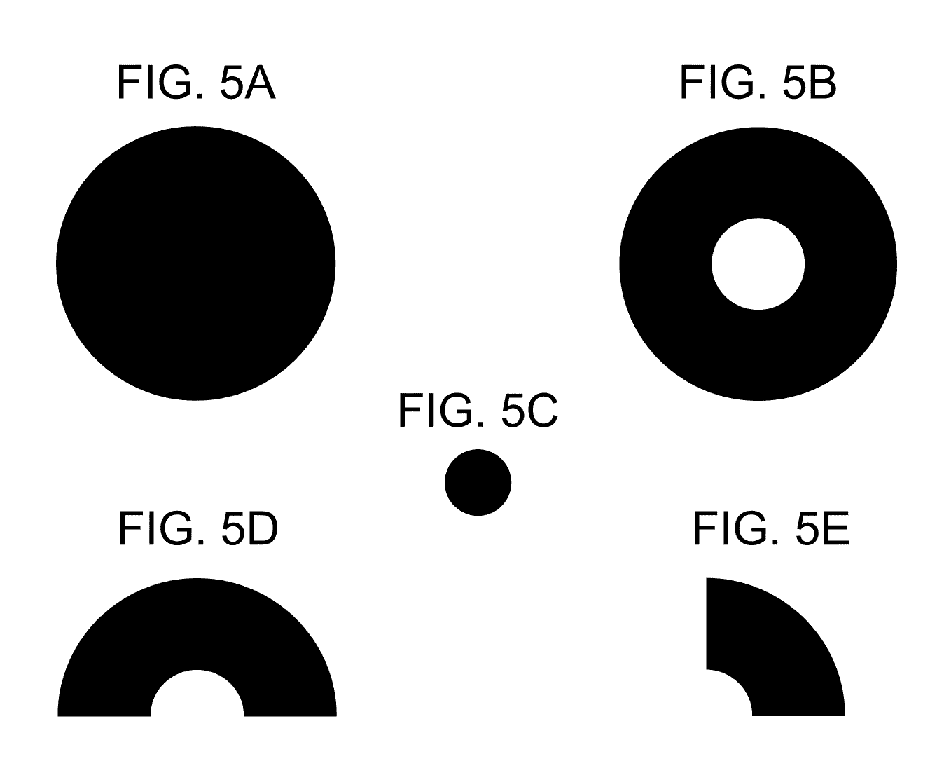

For example, FIGS. 5A-5E show illustrative stimulus patterns, which can be generated by pupillary imaging device 40, according to an embodiment. As described herein, each stimulus pattern can be presented as a flash in a series of flashes. As illustrated, the stimulus patterns can include, for example: a full field pattern shown in FIG. 5A, which is configured to stimulate substantially all of the retina; a ring (macular sparing) pattern shown in FIG. 5B, which is configured to stimulate the retina immediately adjacent to the macula, without stimulating the macular area; a spot pattern shown in FIG. 5C, which is configured to stimulate an area that approximates the macular area of the retina; a superior hemi-field pattern shown in FIG. 5D, which is configured to stimulate a superior portion of the retina, without stimulating the macular area; and a superior quadrant pattern shown in FIG. 5E, which is configured to stimulate a superior nasal portion of the retina, without stimulating the macular area. It is understood that additional and/or alternative patterns can be included in the group of stimulus patterns. For example, the stimulus patterns can include an inferior hemi-field pattern, which is configured to stimulate the inferior portion of the retina, without stimulating the macula. Similarly, the stimulus patterns can include an inferior quadrant pattern, which is configured to stimulate an inferior nasal portion of the retina, without stimulating the macula. Furthermore, it is understood that the patterns can include any superior and/or inferior quadrant pattern.

Furthermore, the series of flashes can include flashes having attributes that vary by one or more of: chromaticity, luminosity, location in the visual field, and/or the like. For example, a series of flashes can include one or more flashes of white, red, green, blue, and/or yellow colors. Similarly, a series of flashes can include multiple subsets of flashes that differ by illuminance. In an embodiment, two illuminances are used, a high intensity illuminance and a low intensity illuminance. For example, a first subset of flashes can comprise a low intensity illuminance of approximately twenty-five lux and a second subset of flashes can comprise a high intensity illuminance of approximately thirty-five lux. However, it is understood that the number of subsets and corresponding illuminances are only illustrative. Additionally, a series of light stimuli can include an analog of the swinging flashlight test in addition to the various patterned stimuli. For example, the analog of the swinging flashlight test can include a series of white light full field patterns, which are presented for approximately one second, followed by a period of no stimulation of approximately 1.6 seconds. In an embodiment, the analog of the swinging flashlight test is presented after the various patterned stimuli in the series of stimuli.

In a more particular illustrative embodiment, an illustrative test series of flashes can comprise: a series (e.g., seven) of high intensity white flashes for each eye of a full field pattern shown in FIG. 5A; a series (e.g., fifteen) of high intensity flashes for each eye of the full field pattern shown in FIG. 5A, each of which varies by color (e.g., white, red, green, blue, yellow); a series (e.g., ten) of color-varying low intensity flashes for each eye of the ring pattern shown in FIG. 5B; a series of color-varying low intensity flashes for each eye of the spot pattern shown in FIG. 5C; a series of color-varying high intensity flashes for each eye of the ring pattern shown in FIG. 5B; a series of color-varying high intensity flashes for each eye of the spot pattern shown in FIG. 5C; a series of color-varying high intensity flashes for each eye of the superior nasal quadrant pattern similar to that shown in FIG. 5E; a series of color-varying high intensity flashes for each eye of an inferior nasal quadrant pattern similar to that shown in FIG. 5E; and a series of high intensity white flashes for each eye of alternating superior and inferior hemi-field patterns similar to that shown in FIG. 5D. After each series, computer system 20 can delay the start of the next series by a predefined rest period, e.g., fifteen seconds. Furthermore, a user 12 can manually select to extend the rest period for any period of time. It is understood that the test series described above is only illustrative. Furthermore, it is understood that in addition to varying by color, the various series of flashes can vary by flash duration, location in the field of view, number of repetitions, and/or the like.

As discussed herein, computer system 20 can process the evaluation data 34 acquired using the series of stimuli in order to evaluate/assist in the evaluation of the patient 2. For example, computer system 20 can process the evaluation data 34 to generate additional evaluation data 34 for the patient 2. In particular, computer system 20 can determine the pupil dimensions of both pupils of the patient 2 during a resting period. Furthermore, computer system 20 can obtain measurements for each pupil corresponding to each stimulus. The measurements can correspond to various variables including: an onset of constriction activity in response to the stimulus, a maximum constriction dimension, dilation recovery, and/or the like. For each variable, the computer system 20 can include one or more measurements indicating dimensional, timing (e.g., latencies), velocity, and/or the like, values for the corresponding variable. The measurements of the variables can be used to create a unique “signature” for each stimulus in the series of stimuli. Furthermore, computer system 20 can perform a direct comparison of a set of parameters of the pupil information for both pupils, such as a constriction amplitude, a constriction latency, a constriction culmination time/velocity, a dilation time/velocity, and/or the like.

Computer system 20 can provide some or all of the evaluation data 34 for use by the user 12 in evaluating the patient 2. For example, computer system 20 can display concurrently acquired image data corresponding to opposing halves of the pupils of the patient 2, which can enable the user 12 to identify differences in the sizes of the pupils. Similarly, computer system 20 can generate and provide any combination of various charts and/or graphs, such as the chart shown in FIG. 4, for presentation to a user 12, which assist the user 12 in comparing and evaluating the responses of both pupils to one or more stimuli.

Furthermore, computer system 20 can evaluate some or all of the evaluation data 34 to determine whether an ocular dysfunction is present in the patient 2. For example, computer system 20 can make a determination of the presence/absence of an ocular dysfunction by evaluating the pupillary responses, e.g., pupil reflexes or defects in such reflexes, to stimuli to the macula and make a comparison to such reflexes or defects in reflexes as seen by stimuli to non-macular areas. Pupil reflexes or defects in reflexes may be seen as being consistent regardless of the stimulated area, changed in magnitude depending on area stimulated, or even reversed as to the eye that presents a defect in reflex. The dysfunction(s) detected may show variable depth and even opposite dysfunctions from macular to non-macular retina responses depending on the presence of any other underlying ocular disorder that is occurring in addition to the macular degeneration, diabetic retinopathy, and/or other pathologies with ocular manifestations being evaluated using the measured pupillary reflexes.

In an embodiment, computer system 20 can determine the pupil sizes, which computer system 20 can use to determine the Relative Afferent Pupillary Defects (RAPD) evoked by each flash. By exposing both eyes to the same series of stimuli, and concurrently recording and measuring the direct and consensual pupillary reflexes for each stimulus, computer system 20 can calculate two values for the RAPD. For example, when the left eye of a patient 2 is exposed to the series of stimuli, computer system 20 can calculate the RAPD for each stimulus by subtracting the direct pupillary reflex of the left eye (OSD) from the consensual pupillary reflex of the right eye (ODC), or ODC-OSD. Second, computer system 20 can calculate the RAPD when the right eye is exposed to the same series of stimuli. In this case, computer system 20 can calculate the RAPD for each stimulus by subtracting the consensual pupillary reflex of the left eye (OSC) from the direct pupillary reflex of the right eye (ODD), or ODD-OSC. Computer system 20 can evaluate a non-zero result for either of the calculations as an indication that an ocular dysfunction is present. The size of the difference provides some indication of the extent of the ocular dysfunction. Furthermore, the sign of the difference indicates the eye in which the defect is present. For example, when the left eye is subtracted from the right eye for each stimulus, a positive value indicates a left afferent defect (LA) and a negative value indicates a right afferent defect (RA).

As described herein, pupillary imaging device 40 can use a series of flashes of light to stimulate pupillary responses from a patient 2, which can subsequently be used to determine the presence of an ocular dysfunction. Using flashes having short time durations (e.g., less than or equal to approximately 0.6 seconds) can allow for a substantial increase in the number of distinct afferent and efferent reflex pathways that can be probed as compared with SFT. Probing a larger number of pathways can allow for a highly discriminative and sensitive measure of any optic neuropathology that can manifest in any of the different conductive ocular pathway pathologies. It further can allow for a separate assessment of efferent pathology. Computer system 20 can process the evaluation data 34 provided by the series of flashes to detect afferent optic nerve or efferent pupillary asymmetry. Furthermore, the evaluation data 34 can provide a direction sensitive measurement of pupillary reflexes in both eyes. Consequently, an embodiment of the invention can provide sufficiently sensitive measurements to evaluate asymmetric precursory manifestations (i.e., ocular dysfunctions) of ocular disorders (e.g., diseases or pathologies) that are bilateral in nature. For example, ocular dysfunctions present in disorders such as the glaucoma group of eye diseases, optic neuritis, retinal pathologies, or other pathologies with ocular manifestations can be detected using an embodiment of the present invention.

Computer system 20 can evaluate the patient 2 for the presence of one or more of various conditions (e.g., disorders, injuries, and/or the like) using the evaluation data 34. For example, computer system 20 can evaluate a difference in the pupil dimensions during rest ocular dysfunction as being indicative of anisocoria. Furthermore, computer system 20 can process the recorded pupillary reflexes to evaluate the presence of an ocular disorder, such as glaucoma, optic neuritis, macular degeneration, and/or the like. Additionally, computer system 20 can process the recorded pupillary reflexes to screen for neurodegenerative diseases, such as multiple sclerosis. Furthermore, computer system 20 can evaluate the measured pupillary reflexes for the presence of diabetic retinopathy or other pathologies with ocular manifestations.

Computer system 20 also can implement a discriminant analysis approach to combine a subset of the collected evaluation data 34 into scores corresponding to one or more ocular dysfunctions, which computer system 20 can use to categorize the patient 2 into one of several clinical categories corresponding to the presence of zero or more conditions. Any clinical category for which RAPD is symptomatic can be identified. Additionally, computer system 20 can use an identified set of test results to create a model, which categorizes future, unknown results. Computer system 20 can apply a hold-out method to cross-validate the model.

Computer system 20 also can implement a multivariate mode of analyses to further discriminate between the various optic dysfunctions of, for example, patients diagnosed with the glaucoma group of diseases, or another ocular disorder and/or neurodegenerative disease. For example, the Pearson product moment correlation coefficients between the matrices of the RAPDs of any number of selected patients' eyes can be calculated so as to determine the extent of the resemblance between the pattern of RAPDs of each of these patients' individual eye or eyes. In order to obtain the best results, the flashes can be provided in the same, or as close to the same as possible, sequence to each patient 2. A high correlation between the ocular dysfunctions of a first patient and a second patient known to have a particular condition can indicate that the first patient also has the condition. When data from numerous patients is used for each condition, computer system 20 can construct a set of inter-correlation matrices. The inter-correlation matrices can provide a user 12 an ability to compare relevant correlations between a test patient's 2 data and that of index patients having various diagnosed conditions. The inter-correlation matrices allow precise quantitative assessment of the resemblance of the data recorded for a test patient 2 to the data recorded for patients clinically diagnosed as having various conditions.

In an embodiment, computer system 20 can process evaluation data 34 to identify individuals having other types of conditions, such as a biomarker of a predisposition of a disorder, such as Alzheimer's. In general, a biomarker is defined as a measure of a biological process or other feature that can be correlated with some aspect of normal body function, an underlying disease process, or a response to a treatment. Based on the analysis to date, an embodiment of the invention can process evaluation data 34 to identify a set of ocular dysfunctions corresponding to a disease trait biomarker and/or a disease state biomarker for Alzheimer's in individuals.

In an embodiment, computer system 20 can evaluate the results to identify whether the patient has an injury-related condition, such as having incurred a traumatic brain injury (TBI). Unlike a neuro-degeneration classification, TBI is a result of an injury, not disease, and is more likely to cause efferent lesions. However, both are similar in that they can interfere with neural conduction pathways of the eyes. Computer system 20 also can use efferent defect information to detect other conditions, such as Horner's Syndrome.

In an embodiment, pupillary evaluation system 10 can be further configured to reduce the stimuli received by one or more other senses of the patient 2 while the visual system is being stimulated and measured. For example, pupillary evaluation system 10 can include an auditory component 42, which can be configured to provide an auditory barrier for the auditory organs of the patient 2 being evaluated. The auditory component 42 can comprise one or more passive and/or active sound blocking components to provide the auditory barrier. For example, the auditory component 42 can comprise ear plugs, head phones, and/or the like, to provide passive sound blocking Additionally, computer system 20 can control the auditory component 42 to generate sounds having noise canceling features while the visual system is being stimulated and measured. Regardless, inclusion of auditory component 42 and/or other external stimuli reducing mechanisms can result in improved measurements of the light-elicited pupillary responses. In particular, a presence of pupillary responses that are the result of non-light stimuli, such as auditory affected pupillary responses, can be reduced in the measurement data.

Pupillary evaluation system 10 can be utilized in an environment, such as at a MASH unit or an emergency room, in which individuals that may have suffered TBI or an ocular injury are likely to seek care. In this case, pupillary evaluation system 10 can be used by healthcare providers to properly test standardized pupil responses, allowing the pupil responses to be periodically quantified and documented in the clinical record. Such monitoring can help avert potentially fatal consequences of increasing intracranial pressure that can accompany evolving cerebral edema or subdural hematoma, subarachnoid hemorrhage induced cerebral vasospasm and ischemia. In situations where ocular motility deficits have been previously elicited, the periodic monitoring of the progression or recovery of those might also help direct timely intervention and proper management of patients 2, such as injured soldiers. Early detection of potential TBI will allow for more effective treatment especially in those cases where cognitive and/or behavioral symptoms do not manifest, or are not easily recognized. Pupillary evaluation system 10 can provide quantitative, automated, and standardized pupillary function, which can assist in correctly identifying TBI, especially where expert neurologists are not available.

While shown and described herein as a method and system for evaluating pupillary responses to light stimuli, it is understood that aspects of the invention further provide various alternative embodiments. For example, in one embodiment, the invention provides a computer program fixed in at least one computer-readable medium, which when executed, enables a computer system to evaluate pupillary responses to light stimuli. To this extent, the computer-readable medium includes program code, such as evaluation program 30 (FIG. 1), which implements some or all of a process described herein. It is understood that the term “computer-readable medium” comprises one or more of any type of tangible medium of expression, now known or later developed, from which a copy of the program code can be perceived, reproduced, or otherwise communicated by a computing device. For example, the computer-readable medium can comprise: one or more portable storage articles of manufacture; one or more memory/storage components of a computing device; paper; and/or the like.

In another embodiment, the invention provides a method of providing a copy of program code, such as evaluation program 30 (FIG. 1), which implements some or all of a process described herein. In this case, a computer system can process a copy of program code that implements some or all of a process described herein to generate and transmit, for reception at a second, distinct location, a set of data signals that has one or more of its characteristics set and/or changed in such a manner as to encode a copy of the program code in the set of data signals. Similarly, an embodiment of the invention provides a method of acquiring a copy of program code that implements some or all of a process described herein, which includes a computer system receiving the set of data signals described herein, and translating the set of data signals into a copy of the computer program fixed in at least one computer-readable medium. In either case, the set of data signals can be transmitted/received using any type of communications link.

In still another embodiment, the invention provides a method of generating a system for evaluating pupillary responses to light stimuli. In this case, a computer system, such as computer system 20 (FIG. 1), can be obtained (e.g., created, maintained, made available, etc.) and one or more components for performing a process described herein can be obtained (e.g., created, purchased, used, modified, etc.) and deployed to the computer system. To this extent, the deployment can comprise one or more of: (1) installing program code on a computing device; (2) adding one or more computing and/or I/O devices to the computer system; (3) incorporating and/or modifying the computer system to enable it to perform a process described herein; and/or the like.

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to an individual in the art are included within the scope of the invention as defined by the accompanying claims.

What is claimed is:

1. A method comprising:
exposing a first eye of a patient to a series of flashes, wherein the series of flashes includes at least one flash corresponding to each of a plurality of patterns, wherein each of the plurality of patterns is configured to stimulate at least one of: a targeted visual function or a targeted cell population of a retina of the first eye; and
concurrently acquiring image data corresponding to the pupillary reflexes of the first eye and a second eye of the patient during the exposing.

2. The method of claim 1, wherein the plurality of patterns includes: at least one elliptical pattern, at least one annular pattern, and at least one partial annular pattern.

3. The method of claim 1, wherein at least some of the plurality of patterns are configured based on at least one of: a size or a location of a macula of the first eye.

4. The method of claim 1, wherein the series of flashes further includes at least one flash having each of a plurality of unique colors.

5. The method of claim 1, wherein the series of flashes further includes at least one flash having each of a plurality of unique illuminances.

6. The method of claim 1, wherein the series of flashes further includes at least one flash located in each of a plurality of locations in the visual field.

7. The method of claim 1, further comprising repeating the exposing and concurrently acquiring for the second eye of the patient.

8. The method of claim 1, further comprising reducing at least one non-visual stimulus experienced by the patient during the exposing.

9. The method of claim 1, further comprising determining a pupillary response of the first eye and the second eye to each of the series of flashes.

10. The method of claim 9, further comprising evaluating the pupillary responses to determine whether an ocular dysfunction is present.

11. The method of claim 10, further comprising evaluating a presence of a condition in the patient in response to evaluating the ocular dysfunction as being present.

12. A system comprising:
a stimulus panel configured to generate a flash of light having any one of a plurality of patterns;
a set of imaging devices configured to concurrently acquire image data of a first eye and a second eye of a patient; and
a computer system for performing a method comprising:
exposing the first eye of the patient to a series of flashes using the stimulus panel, wherein the series of flashes includes at least one flash corresponding to each of the plurality of patterns, wherein each of the plurality of patterns is configured to stimulate at least one of: a targeted visual function or a targeted cell population of a retina of the first eye; and
concurrently acquiring image data corresponding to the pupillary reflexes of the first eye and the second eye of the patient during the exposing using the set of imaging devices.

13. The system of claim 12, wherein the plurality of patterns includes: at least one elliptical pattern, at least one annular pattern, and at least one partial annular pattern, and wherein at least some of the plurality of patterns are configured based on at least one of: a size or a location of a macula of the first eye.

14. The system of claim 12, the method further comprising repeating the exposing and concurrently acquiring for the second eye of the patient.

15. The system of claim 12, further comprising an auditory component configured to provide an auditory barrier for a set of auditory organs of the patient, the method further comprising reducing auditory stimuli experienced by the patient during the exposing using the auditory component.

16. The system of claim 12, wherein the stimulus panel is a liquid crystal display (LCD) panel, and wherein the method further comprises generating a fixation target for the patient using the stimulus panel during the exposing.

17. The system of claim 12, wherein the set of imaging devices comprise infrared-based imaging devices, and wherein the method further comprises illuminating the first eye and the second eye with infrared light during the exposing.

18. The system of claim 12, the method further comprising:
determining a pupillary response of the first eye and the second eye to each of the series of flashes;
evaluating the pupillary responses to determine whether an ocular dysfunction is present; and
evaluating a presence of a condition in the patient in response to evaluating the ocular dysfunction as being present.

19. A system for evaluating a patient, the system comprising:
a stimulus panel configured to generate a flash of light having any one of a plurality of patterns;
a set of imaging devices configured to concurrently acquire image data of a first eye and a second eye of a patient; and
a computer system for performing a method comprising:
exposing the first eye of the patient to a series of flashes using the stimulus panel, wherein the series of flashes includes at least one flash corresponding to each of the plurality of patterns, wherein each of the plurality of patterns is configured to stimulate at least one of: a targeted visual function or a targeted cell population of a retina of the first eye;
concurrently acquiring image data corresponding to the pupillary reflexes of the first eye and the second eye of the patient during the exposing using the set of imaging devices;
repeating the exposing and concurrently acquiring for the second eye of the patient; and
determining a pupillary response of the first eye and the second eye to each of the series of flashes corresponding to the first eye and the second eye.

20. The system of claim 19, the method further comprising providing a graphical representation of the pupillary responses for presentation to a user.

21. The system of claim 19, further comprising an auditory component configured to provide an auditory barrier for a set of auditory organs of the patient, the method further comprising reducing auditory stimuli experienced by the patient during the exposing using the auditory component.

22. The system of claim 19, wherein the plurality of patterns includes: at least one elliptical pattern, at least one annular pattern, and at least one partial annular pattern, and wherein at least some of the plurality of patterns are configured based on at least one of: a size or a location of a macula of the first eye.

23. The system of claim 19, the method further comprising evaluating the pupillary responses to determine whether an ocular dysfunction is present.

24. The system of claim 23, the method further comprising evaluating a presence of a condition in the patient in response to evaluating the ocular dysfunction as being present.
